(19) United States
(12) Patent Application Publication
(10) Pub. No.: US 2024/0082131 A1
(43) Pub. Date: Mar. 14, 2024

(54) COMPOSITION FOR IMPROVING SKIN CONDITIONS

(71) Applicant: NEOPHARM CO., LTD., Daejeon (KR)

(72) Inventors: Yoon KIM, Daejeon (KR); Kyung Sook YOO, Daejeon (KR); Bu-Mahn PARK, Daejeon (KR); Yu Ra JUNG, Daejeon (KR); Hye Seong SHIN, Daejeon (KR); A Ju KIM, Daejeon (KR)

(21) Appl. No.: 18/513,133

(22) Filed: Nov. 17, 2023

Related U.S. Application Data
(62) Division of application No. 17/521,928, filed on Nov. 9, 2021.

(30) Foreign Application Priority Data
Nov. 11, 2020 (KR) 10-2020-0149807

(51) Int. Cl. A61K 8/44; A61Q 17/00; A61Q 19/00

(52) U.S. Cl. CPC A61K 8/442; A61Q 17/005; A61Q 19/007

(57) ABSTRACT

The present invention relates to a cosmetic composition for improving skin conditions that has an effect of enhancing skin defense, improving skin moisturizing, and strengthening a skin barrier, and has an antimicrobial activity. According to the present invention, production and activities of antimicrobial peptides in keratinocytes may be significantly increased, growth of Staphylococcus may be effectively inhibited, and a synergy effect may be imparted to gene expression of a skin moisturizing factor and an intercellular binding factor, such that a synergistic effect in improving skin conditions may be obtained without side effects.

### TECHNICAL FIELD

The present disclosure relates to a composition for improving skin conditions, and more particularly, to a cosmetic composition for improving skin conditions that has an effect of enhancing skin defense, improving skin moisturizing, and strengthening a skin barrier, and has an antimicrobial activity.

### BACKGROUND ART

Exogenous skin diseases have significantly increased due to changes in lifestyle. In particular, in accordance with changes in living environments in recent years, city people have been increasingly exposed to exogenous pathogenic bacteria such as ticks that live in carpets in homes, bacteria and fungi that reproduce depending on humidity and temperature, and viruses in public places. In order to combat these exogenous pathogenic bacteria, the immune system of the skin, which is the outermost organ, of a human body is becoming important.

An example of a substance responsible for an in vivo defense mechanism except for physical defense among defense mechanisms of the skin may include an antimicrobial peptide. The antimicrobial peptide is largely divided into two types such as cathelicidin and defensin. The cathelicidin exhibits a broad antimicrobial activity and has various immunomodulatory functions. In particular, LL-37 is the only known cathelicidin expressed in humans, and production thereof is determined by factors such as the presence of bacteria, secretion of cytokines of a host, the amount of oxygen available in a body, and vitamin D. LL-37 acts to induce a biological immune response to wounds or infections of external tissues, exhibits chemotaxis toward eosinophils, monocytes, and T cells as well as a direct antimicrobial activity against bacteria, fungi, viruses, and the like, and induces proliferation of endothelial cells. In particular, LL-37 present in the skin is responsible for an antigen inhibition function by acting as an immediate defense at the time of penetration of foreign antigens. In addition, defensins are one of the most studied antimicrobial peptides and are largely classified into α-defensin and β-defensin depending on structural characteristics thereof. β-defensin is an antimicrobial peptide expressed in mucous epithelium such as skin, lungs, organs, kidneys, and reproductive organs. Until now, six types of human-derived β-defensins, that is, human β-defensin-1 (hBD-1), human β-defensin-2 (hBD-2), human β-defensin-3 (hBD-3), human β-defensin-4 (hBD-4), human β-defensin-5 (hBD-5), and human β-defensin-6 (hBD-6) have been separated and identified. In particular, it is known that hBD-1 is constitutively expressed in epidermal cells, whereas hBD-2 is increasingly expressed at an infected region or a physically damaged region and plays an important role in controlling a systemic immune response and an inflammatory response. In addition, it has been reported that hBD-3 is significantly highly expressed at skin lesion regions of psoriasis patients, and it is known that hBD-3 is inducibly expressed by cytokines and a bacterial product. hBD-4 exhibits a more restricted distribution compared to hBD-1, hBD-2, or hBD-3, and expression thereof may be upregulated by infection of bacteria, but hBD-4 is not upregulated by inflammatory factors that upregulate hBD-2 and hBD-3. hBD-5 and hBD-6 are the most recently identified and are family members primarily present in epithelium. In addition, recently, β-defensin is known to be involved not only in local infection defense but also in acquired immune by chemotactic migration of cells such as dendritic cells, T lymphocytes, and monocytes.

Meanwhile, sensitive skin is likely to be affected by skin immunological diseases such as atopic dermatitis and psoriasis when being exposed to harmful environments. In the related art, the skin immunological diseases such as atopic dermatitis and psoriasis are mainly relieved by a method of maintaining moisture by using ceramides; however, a moisturizing power decreases over time and the skin conditions return to the original state. Accordingly, a method of producing antimicrobial peptides from the outside and injecting the antimicrobial peptides into the skin may be considered, but the antimicrobial peptides do not easily directly penetrate into the skin and there is a risk of an autoimmune reaction or the like. Therefore, a method of promoting production of antimicrobial peptides by skin cells themselves may be a method of enhancing skin immunity from the outside.

Under such a background, research on a method of promoting production and activities of antimicrobial peptides has been actively conducted, but remarkable research results are not yet known. Therefore, there is an urgent need for a development of a substance that is safe when applied to a human body, has stable active ingredients, and above all, has an effect of enhancing skin defense and improving skin moisturizing and has an excellent antimicrobial activity through improvement of skin immunity and enhancement of an antimicrobial activity against atopic dermatitis, psoriasis, and the like, compared to existing substances.

### DISCLOSURE

### Technical Problem

An embodiment of the present invention is directed to providing a cosmetic composition for improving skin conditions that has an effect of enhancing skin defense, improving skin moisturizing, and strengthening a skin barrier, and has an antimicrobial activity.

### Technical Solution

In one general aspect, a cosmetic composition for enhancing skin defense contains N-(1-oxodecyl)serine alkyl ester as an active ingredient.

The active ingredient may be N-(1-oxodecyl)serine methyl ester, N-(1-oxodecyl)serine ethyl ester, or a combination thereof.

The skin defense may be enhanced by promoting expression of an antimicrobial peptide in skin.

The antimicrobial peptide may be selected from hBD-2, hBD-3, and LL-37.

In another general aspect, a cosmetic composition for exhibiting an antimicrobial effect contains N-(1-oxodecyl)serine alkyl ester as an active ingredient.

The antimicrobial effect may be exhibited by inhibiting growth of Staphylococcus; and promoting expression of an antimicrobial peptide in skin.

The Staphylococcus may be selected from i Staphylococcus aureus, Staphylococcus epidermidis, and Staphylococcus pyogenes.

In still another general aspect, a cosmetic composition for skin moisturizing contains N-(1-oxodecyl)serine alkyl ester as an active ingredient.

The cosmetic composition may exhibit a skin moisturizing effect by increasing a skin moisturizing factor.

The skin moisturizing factor may be selected from filaggrin and loricrin.

In further still another general aspect, a cosmetic composition for strengthening a skin barrier contains N-(1-oxodecyl)serine alkyl ester as an active ingredient.

The active ingredient may be contained in an amount of 0.001 to 5 wt % with respect to a total weight of the cosmetic composition.

### Advantageous Effects

According to the present invention, it is possible to significantly increase production and activities of antimicrobial peptides in keratinocytes. Therefore, the skin defense may be enhanced through the improvement of skin immunity and antimicrobial effect. In addition, the growth inhibition and destroying effect on Staphylococcus may be excellent. With these effects, according to the present invention, the overall skin conditions may be improved, and the effective effects on exogenous skin diseases caused by various causes may be exhibited.

When the cosmetic composition of the present invention is applied to a human body, the expression of the skin moisturizing factors may be promoted together with the activities of the antimicrobial peptides as described above, and the skin barrier damaged by ultraviolet rays may be efficiently strengthened. Therefore, the cosmetic composition of the present invention helps the skin tissues to stably maintain moisture and strengthens the skin barrier, such that the skin moisturizing effect to maintain the moisture in the skin may be excellent. In addition, the cosmetic composition of the present invention may be applied as an external skin formulation such as pharmaceuticals and cosmetics to exhibit effective effects because it is safe for a human body and has no side effects even after long-term use.

### DESCRIPTION OF DRAWINGS

FIG. 1 is a graph showing a comparison of effects of Examples and Comparative Example of the present invention on expression of antimicrobial peptides (LL-37) in skin.

FIG. 2 is a graph showing a comparison of effects of Examples and Comparative Example of the present invention on expression of antimicrobial peptides (BD-2) in skin.

FIG. 3 is a graph showing a comparison of effects of Examples and Comparative Example of the present invention on expression of antimicrobial peptides (BD-3) in skin.

FIG. 4 is a graph showing results of inhibition of growth of Staphylococcus aureus in Example and Comparative Example of the present invention.

FIG. 5 is a graph showing a comparison of results of expression of filaggrin genes after being treated with samples of Examples and Comparative Example of the present invention with results of a control and a UVB treatment group.

FIG. 6 is a graph showing a comparison of effects of Examples and Comparative Example of the present invention on expression of desmoglein1 (DSG1) gene as an intercellular binding (desmosome) factor.

### BEST MODE

A composition for improving skin conditions according to the present invention will be described below. Technical terms and scientific terms used herein have the general meanings understood by those skilled in the art to which the present invention pertains unless otherwise defined, and a description for the known function and configuration unnecessarily obscuring the gist of the present invention will be omitted in the following description.

The term “improvement” used in the present invention refers to any action in which skin conditions are improved or beneficially changed by application of a cosmetic composition according to the present invention.

The term “filaggrin” used in the present specification is a keratin-binding protein isolated from mammalian epidermal cells and refers to a basic protein having a molecular weight of about 260,000. It is known that the protein binds to a keratin unit in a ratio of about 3:2 to form fibers, the fibers are aggregated in the form of bundles to form macrofibrils, the macrofibrils undergo cleavage by protease and dephosphorylation to form filaggrin when accumulated in cells to differentiate as a highly phosphorylated precursor having a molecular weight of about 500,000.

The term “loricrin” used in the present specification refers to loricrin that is expressed while undergoing a final differentiation and may be used as a marker in tracking a final differentiation process of keratinocytes because it binds to a cell membrane at an upper stratum granulosum to complete a protein.

The present inventors have intensively conducted studies on a cosmetic material capable of activating an antimicrobial peptide and have confirmed that an amide-based compound having a specific structure may simultaneously activate cathelicidin and defensin. Specifically, the amide-based compound according to the present invention may promote an activity of an antimicrobial peptide such as LL-37, hBD-2, or hBD-3.

The present inventors have particularly focused on N-(1-oxodecyl)serine alkyl ester with such an approach. The N-(1-oxodecyl)serine alkyl ester promotes the activity of the antimicrobial peptide described above and has an excellent antimicrobial activity against bacteria, such that defense of the skin itself may be enhanced. When bacteria invade the skin, the skin secretes antimicrobial peptides to combat bacteria itself. Therefore, the promotion of the activity of the antimicrobial peptide means that invading bacteria may be more effectively suppressed. Thus, exogenous skin diseases such as acne may be effectively relieved. In addition, the N-(1-oxodecyl)serine alkyl ester may increase, supplement, and maintain various skin moisturizing factors and may increase expression of intercellular binding factor genes, such that a skin barrier function may be enhanced. Accordingly, the present inventors intend to suggest the present invention by revealing the use of the N-(1-oxodecyl)serine alkyl ester, which has not previously been known, based on such effects.

Hereinafter, the present invention will be described in detail.

A cosmetic composition of the present invention contains N-(1-oxodecyl)serine alkyl ester as an active ingredient, and a specific function thereof is as follows.

A first aspect of the present invention is a cosmetic composition for enhancing skin defense.

The cosmetic composition for enhancing skin defense of the present invention enhances skin defense by promoting expression of antimicrobial peptides in the skin. Therefore, the cosmetic composition of the present invention may promote the activities of the antimicrobial peptides in the skin and may also enhance the skin's own immunity without irritating the skin, such that the skin conditions may be improved. On the other hand, an antimicrobial peptide activation effect is not exhibited or insignificantly exhibited in a compound having structural characteristics similar to those of the cosmetic composition of the present invention.

In the cosmetic composition for enhancing skin defense according to an exemplary embodiment of the present invention, the antimicrobial peptide may be selected from LL-37, hBD-2, and hBD-3.

A second aspect of the present invention is a cosmetic composition for exhibiting an antimicrobial effect.

The cosmetic composition for exhibiting an antimicrobial effect of the present invention imparts a significant synergistic effect to the antimicrobial activity by inhibiting growth of Staphylococcus and promoting the activity of the antimicrobial peptide. Therefore, when the cosmetic composition of the present invention is simulated from the outside, expression may be induced to inhibit and destroy the invading bacteria.

The cosmetic composition for exhibiting an antimicrobial effect according to an exemplary embodiment of the present invention exhibits an excellent antimicrobial effect against Staphylococcus selected from Staphylococcus aureus, Staphylococcus epidermidis, and Staphylococcus pyogenes. Examples of diseases caused by Staphylococcus include abscess, acne, localized scratching dermatitis, allergic contact dermatitis, Rhus dermatitis, atopic dermatitis, seborrheic dermatitis, systemic scratching dermatitis, stasis dermatitis, perioral dermatitis, and psoriasis. According to the present invention, these dermatitides may be effectively relieved.

A third aspect of the present invention is a cosmetic composition for skin moisturizing.

The cosmetic composition for skin moisturizing of the present invention promotes expression of skin moisturizing factors such as filaggrin and loricrin to remarkably enhance a moisture retention ability of skin tissues and strengthen a skin barrier when applied to the skin, thereby imparting a synergistic effect to these effects.

A fourth aspect of the present invention is a cosmetic composition for strengthening a skin barrier.

The cosmetic composition for strengthening a skin barrier of the present invention may strengthen the skin barrier by preventing damage of the skin barrier, in particular, by light sources (for example, UVA, UVB, blue light, and the like) and reducing a moisture loss of the skin. Specifically, it is confirmed in skin damaged by ultraviolet rays that expression of keratinocyte differentiation marker genes in the skin is reduced or expression of intercellular binding factor genes is reduced. On the other hand, according to the present invention, the expression of the intercellular binding factor genes is increased to effectively strengthen the skin barrier damaged by ultraviolet rays.

The cosmetic composition of the present invention implements the effects described above at the same time, such that the expression of the antimicrobial peptides in the keratinocytes is increased, thereby enhancing the skin defense, improving skin moisturizing, and strengthening the skin barrier through improvement of skin immunity and enhancement of an antimicrobial activity. As a result, it is possible to provide a more increased skin improvement effect.

With these effects, the cosmetic composition of the present invention described above may be used for a method of improving skin conditions by applying the cosmetic composition to the skin. The improvement may be realized from one or more effects selected from effects obtained by the promotion of the activity of the antimicrobial peptide, the inhibition of growth of Staphylococcus, the strengthening of the skin barrier, and the increase of the skin moisturizing factor.

As described above, the cosmetic composition of the present invention contains N-(1-oxodecyl)serine alkyl ester, which is an amide-based compound, as an active ingredient. Specifically, the N-(1-oxodecyl)serine alkyl ester may be alkyl ester having 4 or fewer carbon atoms.

The N-(1-oxodecyl)serine alkyl ester according to an exemplary embodiment of the present invention may be selected from N-(1-oxodecyl)serine methyl ester and N-(1-oxodecyl)serine ethyl ester represented by the following structures, and the cosmetic composition may contain one or two selected from N-(1-oxodecyl)serine methyl ester and N-(1-oxodecyl)serine ethyl ester.

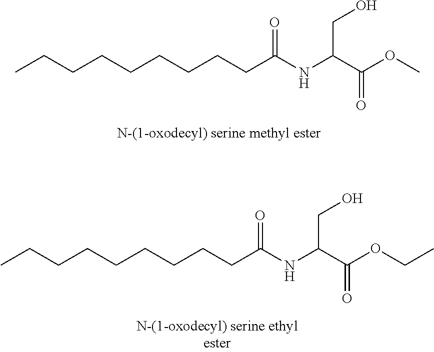

As an example, the cosmetic composition of the present invention may also contain, as an active ingredient, a pharmaceutically acceptable salt, solvate, or stereoisomer of the N-(1-oxodecyl)serine alkyl ester, as an aspect of the present invention.

The N-(1-oxodecyl)serine alkyl ester may be safely used in an amount at which an effective effect on the skin is exhibited because it does not cause cytotoxicity and skin irritation. In addition, the N-(1-oxodecyl)serine alkyl ester implements stable maintenance of its efficacy in a formulation, does not cause a phenomenon of precipitation or separation in the formulation, and has excellent storage stability.

In the cosmetic composition according to an exemplary embodiment of the present invention, the active ingredient may be contained in an amount of 0.001 to 5 wt % with respect to a total weight of the cosmetic composition. The active ingredient is preferably contained in an amount of, specifically, 0.001 to 3 wt %, and more specifically, 0.01 to 1 wt %, with respect to the total weight of the cosmetic composition. When the amount of active ingredient contained is within the above range, the stability of the formulation is not impaired and the desired effects in the present invention are obtained, that is, the use thereof is remarkably effective, which is preferable.

In addition, in the cosmetic composition according to an exemplary embodiment of the present invention, when the active ingredient is contained in a form of a mixture, a more remarkable synergistic effect may be imparted, which is preferable. In particular, in a case where a combination of the N-(1-oxodecyl)serine alkyl ester is contained as an active ingredient, expression of a skin moisturizing factor and desmoglein1 (DSG1) gene is remarkably promoted.

As an example, when an active ingredient in which the N-(1-oxodecyl)serine methyl ester and the N-(1-oxodecyl)serine ethyl ester are mixed (1:1, weight ratio) is contained, the effect is further increased compared to a case of containing a single component used in the same amount as that of the active ingredient.

As an example, in an active ingredient in which the N-(1-oxodecyl)serine methyl ester and the N-(1-oxodecyl)serine ethyl ester are mixed, the N-(1-oxodecyl)serine methyl ester and the N-(1-oxodecyl)serine ethyl ester may be mixed in a weight ratio of 0.01:99.99 to 99.99:0.01, specifically, in a weight ratio of 1:9 to 9:1, and more specifically, in a weight ratio of 1:1 to 9:1, but the present invention is not limited thereto.

The cosmetic composition according to an exemplary embodiment of the present invention may contain the active ingredient and a balance of water, and may be formulated in various forms.

The cosmetic composition according to an exemplary embodiment of the present invention may be formulated into a general emulsified formulation and a solubilized formulation using a conventionally known preparation method.

As an example, the cosmetic composition may be formulated into a formulation selected from a skin lotion, an astringent lotion, a nourishing lotion, an eye cream, a nourishing cream, a massage cream, a cleansing cream, a cleansing foam, a cleansing water, an essence, and a pack, but the present invention is not limited thereto.

In addition, the cosmetic composition may further appropriately contain additional additives depending on a purpose, and may further contain a component selected from a component for preventing wrinkles, an antioxidant component, and a whitening component known in the art, together with the amide-based compound. As an example, the component may be selected from retinoic acid, a transforming growth factor (TGF), an animal placenta-derived protein, betulinic acid, and a chlorella extract, but the present invention is not limited thereto.

In addition, the cosmetic composition may further contain one or more aqueous additives selected from a stabilizer, an emulsifier, a thickener, a moisturizer, a liquid crystal membrane strengthening agent, a pH regulator, an antimicrobial agent, a water-soluble polymer, a coating agent, a metal ion sequestering agent, an amino acid, an organic amine, a polymer emulsion, a pH adjuster, a skin nutrient, an antioxidant, an antioxidant aid, a preservative, and flavoring; and one or more oil additives selected from oils, waxes, a hydrocarbon oil, a higher fatty acid oil, a higher alcohol, a synthetic ester oil, and a silicone oil.

In this case, each of the additives may be contained in an amount of 0.001 to 20 wt %, specifically, 0.01 to 10 wt %, and more specifically, 0.05 to 5 wt %, with respect to the total weight of the cosmetic composition, but the present invention is not limited thereto.

### (Evaluation Methods)

### 1. Confirmation of Effect of Enhancing Skin Defense

In order to confirm the effect of enhancing the skin immunity and defense of each of Examples and Comparative Example of the present invention, the activities of peptide LL-37 (cathelicidin-related antimicrobial peptide (CAMP)), human β-defensin-2 (hBD-2), and human β-defensin-3 (hBD-3) having antimicrobial efficacy were confirmed.

In order for the confirmation, human epidermal keratinocytes (NHEKs) were dispensed into a 96-well plate, and the cells were cultured under cell culture conditions for 24 hours. Thereafter, the cells were treated with a control, DMSO, and a 1% sample (in H₂O) of each of Examples and Comparative Example, and the cells were additionally cultured for 24 hours. A real-time PCR method was performed to determine LL-37, BD-2, and BD-3 at a gene-level. The test procedure is as follows.

SuperPrep™ cell lysis and RT Kit for qPCR (TOYOBO, SCQ-101) were used for RNA isolation and cDNA synthesis. The cells from which the medium was removed were washed once with phosphate-buffered saline (PBS), 50 μl of a cell lysis mixture (including gDNA remover) was added and reacted for 5 minutes, and then, a stop solution was added. 8 μl of the extracted mRNA was added to 32 μl of a RT reaction mixture, and cDNA was synthesized using PCR at 37° C. for 15 minutes, 50° C. for 5 minutes, and 95° C. for 5 minutes. In order to compare and analyze the expression of genes, the synthesized cDNA was used as a template, and Real-time PCR analysis was performed using Thunderbirds™ SYBR qPCR Mix (TOYOBO, QPS-201). QuantiTect primer assays of Qiagen were used in the experiment as a primer, and the expression levels of LL-37, BD-2, and BD-3 in the samples were quantified as GADPH and compared with each other. In Real-time qPCR analysis, the condition was set so that, first, the reaction was performed at 95° C. for 15 minutes, and then, the reaction was performed at 94° C. for 15 seconds, 60° C. for 30 seconds, and 72° C. for 30 seconds as one cycle, and 40 cycles in total were performed. In this case, N-(1-oxodecyl)serine ethyl ester was used in Example 1, N-(1-oxodecyl)serine methyl ester was used in Example 2, defensamide (Chemfuture) was used in Comparative Example 1, the sample was not treated in the control, and DMSO was a group treated with only the solvent in which the sample was dissolved.

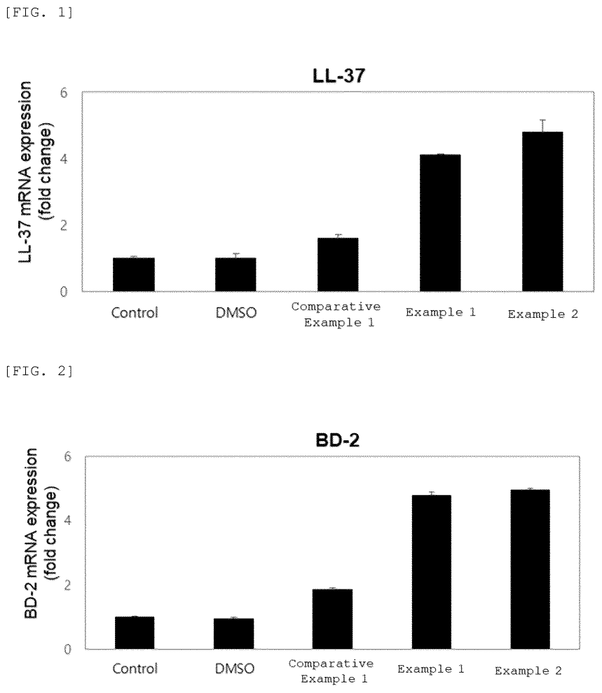

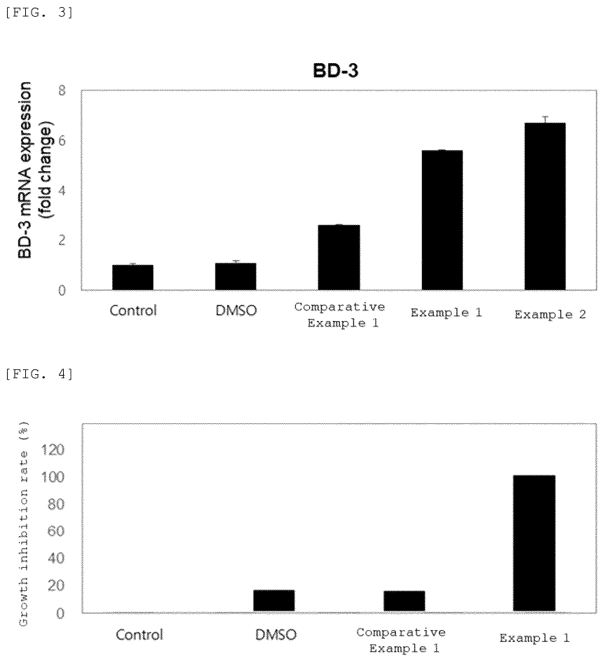

The results obtained therefrom are illustrated in FIGS. 1 to 3.

### 2. Confirmation of Effect of Inhibiting Growth of Staphylococcus

In order to confirm the effect of inhibiting the growth of Staphylococcus aureus in each of Examples and Comparative Example of the present invention, Staphylococcus aureus KCCM12256 supplied by Korean Culture Center of Microorganisms (KCCM) was used. In a Staphylococcus medium, M9 salt was used as a base, and 2 mM MgSO₄, 0.1 mM CaCl₂, 1% (w/v) glucose, 1% (w/v) casamino acid, 1 mM thiamine-HCl, 0.05 mM nicotinamide were added. The thiamine-HCl solution and the nicotinamide solution were filtered using a filter having a pore size of 0.2 μm, and the remaining components were used after being autoclaved at 121° C. for 15 minutes. The respective components in the medium were mixed according to the concentrations, and the mixed component was used after being filtered again using a filter having a pore size of 0.2 μm.

In the growth inhibition experiment, S. aureus was subcultured after seed culture at 37° C. and 200 rpm for 24 hours. The S. aureus was cultured by the subculture up to the mid-exponential phase (OD₆₀₀=2.5), the initial number of bacteria was adjusted to 0.01 at an absorbance of 600 nm in the liquid medium containing the sample of each of Examples and Comparative Example at a concentration of 0.03 mg/ml, 200 μl of the bacteria were dispensed into and cultured in a 96-well plate, and then, an absorbance was measured. The culture and the absorbance measurement were performed using Synergy HTX Multi-Mode Reader of BioTek Instruments. The culture was performed at 37° C. and 200 rpm and the absorbance was measured at a wavelength of 600 nm.

The results obtained therefrom are illustrated in FIG. 4.

### 3. Analysis of Expression Levels of Filaggrin and Loricrin

In order to measure the effect of promoting the expression of moisturizing factors of each of Examples and Comparative Example of the present invention, the expression levels of filaggrin and loricrin were measured.

Specifically, keratinocytes purchased from Invitrogen Corporation were cultured in EpiLife (keratinocyte growth medium) containing a supplement (human keratinocyte growth supplement (HKGS)) for a predetermined time, and then, the cells were subcultured three times and then used. The keratinocytes which were subcultured three times were cultured to a confluence of 70 to 80% in a 6-well plate, each of the wells was separately treated with each of the samples, and then, the keratinocytes were cultured for a total of 24 hours. RNA of the keratinocytes was extracted from the cultured cells using easy BLUE, and then, cDNA synthesis was performed using SuperScript reverse transcriptase III kit. Real-time PCR for comparing the genes was performed using 2× TagMan universal PCR mixture (10 μl), 20× TaqMan expression assay mix (1 μl), cDNA (50 ng), and a primer (Filaggrin: Hs00856927_g1*, Loricrin: Hs01894962_s1*) and a 7500 Fast Real-Time PCR system. In addition, a sample treated with 10 μM IGF-1 was used as a positive control group, and a control that was not treated with the sample was used as a comparison group.

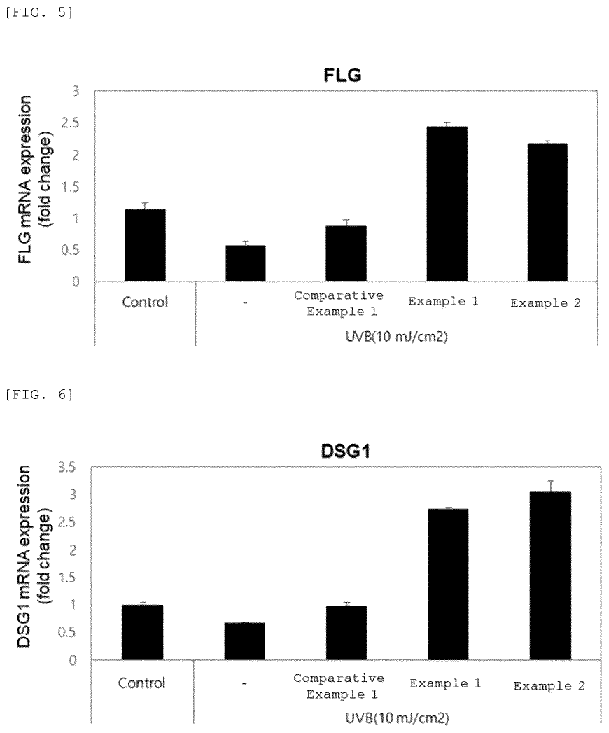

The results obtained therefrom are illustrated in FIG. 5.

### 4. Confirmation of Increase of Expression of Desmoglein1 (DSG1) Gene as Intercellular Binding Factor

In order to confirm the effect of strengthening the skin barrier of each of Examples and Comparative Example of the present invention, an expression level of DSG1 gene as an intercellular binding factor was confirmed.

Specifically, normal human epidermal keratinocytes (NHEKs) were cultured in a 6-well plate incubator. After 24 hours, the keratinocyes were washed with phosphate-buffered saline (PBS), irradiation with UVB (25 mJ/cm²) was performed, a 10 nM sample of each of Examples and Comparative Example was added to the NHEK culture medium, and then, culture was performed. The cells were harvested on the 4^{th} day of culture, RNA was separated, cDNA was synthesized by a reverse transcriptional polymerase chain reaction (RT-PCR), Taqman real-time PCR was performed using the synthesized cDNA, and then, the expression level of the DSG1 gene as a keratinocyte differentiation marker was measured.

The results obtained therefrom are illustrated in FIG. 6.

### EXAMPLES 1 AND 2 AND COMPARATIVE EXAMPLE 1

The evaluation methods described above were performed using the compounds having the structures shown in Table 1.

TABLE 1
Example 1

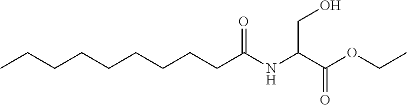 [SMILES: CCCCCCCCCC(=O)NC(CO)C(=O)OCC]

Example 2

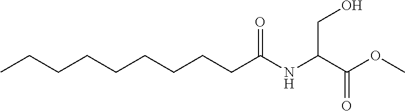 [SMILES: CCCCCCCCCC(=O)NC(CO)C(=O)OC]

Comparative Example 1

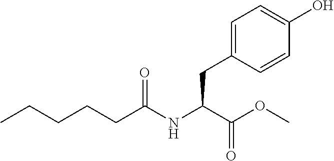 [SMILES: CCCCCC(=O)N[C@@H](Cc1ccc(O)cc1)C(=O)OC]

Example 1: N-(1-oxodecyl)serine ethyl ester
Example 2: N-(1-oxodecyl)serine methyl ester
Comparative Example 1: Defensamide
Control: Treatment group without being subjected to sample treatment
DMSO: DMSO treatment group
-: UVB treatment group

As illustrated in FIGS. 1 to 3, it was confirmed that in the sample treated with the compound of the present invention, the expression of all of the antimicrobial peptides BD-2, BD-3, and LL-37 in skin was increased. On the other hand, in Comparative Example 1, the expression levels of the antimicrobial peptides BD-2, BD-3, and LL-37 in skin were 33%, 40%, and 40%, respectively, relative to those in Example 2, which showed that the expression of the antimicrobial peptide in skin was insignificant. It could be appreciated from the results that in Examples in which the sample was treated with the compound of the present invention, the antimicrobial peptides in skin were activated, and thus, the defense of the skin itself was enhanced and the effect thereof was excellent even in comparison with Comparative Example 1 in which the compound having similar structural characteristics to those of the compound of the present invention.

In addition, this effect tended to be enhanced depending on the concentration.

As illustrated in FIG. 5, it was confirmed that in all of the samples treated with the compound of the present invention, the expression of each of the filaggrin and loricrin was promoted. In particular, it was confirmed that in the case where the sample of Example 1 or 2 was treated, the expression level of the filaggrin was increased by 4.3 times or 3.8 times or more the expression level in the control. This effect corresponds to a significantly improved effect compared to the sample treated with the compound of Comparative Example 1.

As illustrated in FIG. 6, it was confirmed that in all of the samples treated with the compound of the present invention, the expression of desmoglein1 was promoted. In particular, it was confirmed that in the case where the sample of Example 1 or 2 was treated, the expression level of the desmoglein1 was increased by 4.1 times or 4.5 times or more the expression level in the UVB treatment group.

Hereinabove, although the present invention has been described by specific matters and limited exemplary embodiments, they have been provided only for assisting in the entire understanding of the present invention. Therefore, the present invention is not limited to the exemplary embodiments. Various modifications and changes may be made by those skilled in the art to which the present invention pertains from this description.

Therefore, the spirit of the present invention should not be limited to these exemplary embodiments, but the claims and all modifications equal or equivalent to the claims are intended to fall within the scope and spirit of the present invention.

1.-12. (canceled)

13. A method of using a cosmetic composition, comprising N-(1-oxodecyl)serine alkyl ester to enhance skin defense comprising:
applying the cosmetic composition comprising N-(1-oxodecyl)serine alkyl ester to skin; and
promoting expression of an antimicrobial peptide in the skin.

14. The method of claim 13, wherein the active ingredient in the cosmetic composition is N-(1-oxodecyl)serine methyl ester, N-(1-oxodecyl)serine ethyl ester, or a combination thereof.

15. The method of claim 13, wherein the antimicrobial peptide is selected from hBD-2, hBD-3, and LL-37.

16. A method of using a cosmetic composition comprising N-(1-oxodecyl)serine alkyl ester to exhibit an antimicrobial effect comprising:
applying the cosmetic composition comprising N-(1-oxodecyl)serine alkyl ester to skin thereby inhibiting growth of Staphylococcus; and
promoting expression of an antimicrobial peptide in the skin.

17. The method of claim 16, wherein the Staphylococcus is selected from Staphylococcus aureus, Staphylococcus epidermidis, and Staphylococcus pyogenes.

18. A method of using a cosmetic composition comprising N-(1-oxodecyl)serine alkyl ester to moisturize skin comprising:
applying the cosmetic composition comprising N-(1-oxodecyl)serine alkyl ester to skin; and
promoting the expression of a skin moisturizing factor.

19. The method of claim 18, wherein the skin moisturizing factor is selected from filaggrin and loricrin.

20. A method of using a cosmetic composition, comprising N-(1-oxodecyl)serine alkyl ester to strengthen a skin barrier by comprising:
applying a cosmetic composition comprising N-(1-oxodecyl)serine alkyl ester to the skin;
thereby preventing damage of the skin barrier.

21. The method of claim 13, wherein N-(1-oxodecyl)serine alkyl ester is contained in an amount of 0.001 to 5 wt. % with respect to a total weight of the cosmetic composition.

22. The method of claim 16, wherein N-(1-oxodecyl)serine alkyl ester is contained in an amount of 0.001 to 5 wt. % with respect to a total weight of the cosmetic composition.

23. The method of claim 18, wherein N-(1-oxodecyl)serine alkyl ester is contained in an amount of 0.001 to 5 wt. % with respect to a total weight of the cosmetic composition.

26. The method of claim 20, wherein N-(1-oxodecyl)serine alkyl ester is contained in an amount of 0.001 to 5 wt. % with respect to a total weight of the cosmetic composition.
